(19) United States
(12) Patent Application Publication
(10) Pub. No.: US 2011/0106173 A1
(43) Pub. Date: May 5, 2011

(54) Anchor Assembly With Directionally Controlled Saddle Adjustment And Transversely Adjustable Receiver

(75) Inventors: Gary S. Lindemann, Collierville, TN (US); William Benoist, Germantown, TN (US); Charles Anthony Dickinson, Bartlett, TN (US)

(73) Assignee: Warsaw Orthopedic, Inc., Warsaw, IN (US)

(21) Appl. No.: 12/609,983

(22) Filed: Oct. 30, 2009

(51) Int. Cl. A61B 17/86

(52) U.S. Cl. 606/302; 606/305

(57) ABSTRACT

A bone anchor assembly includes a receiver extending along a central longitudinal axis between a proximal end and an opposite distal end and a bone anchor extending from the receiver. The receiver includes a distal portion defining a receptacle opening at the distal end and a pair of arms extending from the distal portion along the central longitudinal axis on opposite sides of a passage. The receiver includes a saddle that is adjustable in the receiver in one plane to accommodate the angle of placement of the connecting element through the receiver. The receiver is adjustable in another plane that is transverse to the adjustment plane of the saddle to better position the receiver to receive the connecting element. The adjustable saddle and receiver reduce the bending or contouring of the connecting element that may be required to position the connecting element in the receiver.

### BACKGROUND

The present invention concerns bone anchors and anchor assemblies, particularly useful for engagement to vertebrae. In a particular embodiment, the invention contemplates a bone anchor assembly with an adjustable saddle and transversely adjustable receiver to secure an elongate connecting element, such as a spinal rod, along the spinal column.

Several techniques and systems have been developed for correcting and stabilizing the spine and for facilitating fusion at various levels of the spine. In one type of system, an elongated rod is disposed longitudinally along the length of the spine or several vertebrae of the spinal column. The rod may be bent to correspond to the normal or desired curvature of the spine in the particular region being instrumented. For example, the rod can be bent or angled to form a normal kyphotic curvature for the thoracic region of the spine, or a lordotic curvature for the lumbar region. In accordance with such a system, the rod is engaged to various vertebrae along the length of the spinal column by way of a number of fixation elements. A variety of fixation elements can be provided which are configured to engage specific portions of the vertebra. For instance, one such fixation element is a hook that is configured to engage the lamina of the vertebra. Another type of fixation element is a spinal screw which can be threaded into various aspects of the vertebral bone, such as the pedicle.

In one typical procedure utilizing a bendable, angled or linear rod, one or more of the rods is situated on one or both of the opposite sides of the spine or spinous processes. A plurality of bone screws are threadingly engaged to several vertebral bodies, such as to the pedicles of these vertebrae. One or more of the bone screws are maneuvered to manipulate the position or orientation of the vertebral body or bodies to which the bone screw is engaged. The rod(s) are connected or affixed to the plurality of bone screws to apply and maintain corrective and stabilizing forces to the spine.

The bone anchors in spinal procedures can have receivers with channels for the elongated rod or other member that, in some bone anchors, open upward, i.e. directly away from the bone to which the anchor is attached. Other bone anchors utilize channels that open along the medial or lateral side of the anchor to receive the rod. It is desirable in some procedures to utilize a bone anchor where the bone engaging portion of the bone anchor and the receiver are fixed relative to one another so that the forces applied to the receiver are effectively transferred to the vertebra. However, the relative positions of the vertebra and the receiver of the bone anchor may require contouring, bending, and/or angling of the rod through the channel of the bone anchor, which can result in a less than optimal fit between the anchor and the rod, creating undesirable stress concentrations in the rod, bone anchor and/or bony structure. Additional improvements in the bone anchor and rod interface in spinal systems are still needed.

### SUMMARY

According to one aspect a bone anchor assembly is disclosed that includes a receiver extending along a central longitudinal axis between a proximal end and an opposite distal end and a bone anchor extending from the receiver. The receiver includes a distal portion defining a receptacle opening at the distal end and a pair of arms extending from the distal portion along the central longitudinal axis on opposite sides of a passage. The receiver includes a saddle that is adjustable in the receiver in one plane to accommodate the angle of placement of the connecting element through the receiver. The receiver is adjustable in another plane that is transverse to the adjustment plane of the saddle to better position receiver to receive the connecting element. The adjustable saddle and receiver reduce the bending or contouring of the connecting element that may be required to position the connecting element in the receiver.

According to another aspect, a bone anchor assembly includes a receiver extending along a central longitudinal axis between a proximal end and an opposite distal end. The receiver includes a distal portion defining a receptacle opening distally and a pair of arms extending from the distal portion along the central longitudinal axis on opposite sides of a passage. The receiver includes a bottom surface extending along the passage between the pair of arms and the passage opens at opposite sides of the receiver between the pair of arms and the receptacle opens into the passage through the bottom surface. The bone anchor assembly also includes a bone anchor including a distal bone engaging portion extending along a longitudinal axis and a head at a proximal end of the distal bone engaging portion that is positioned in the receptacle of the receiver. The bone anchor assembly further includes a saddle positioned in the passage of the receiver adjacent to the bottom surface of the receiver. The saddle includes a proximal support surface and a distal surface opposite the proximal support surface. A connecting element extending along a central longitudinal axis is located in the passage and extends through the opposite sides of the receiver. The saddle engages the connecting element and is limited to movement in the receiver in a first plane defined by the central longitudinal axis of the receiver and the central longitudinal axis of the connecting element while the receiver is pivotal about the head of the bone anchor in a second plane that is orthogonal to the first plane in which the saddle pivots.

According to another aspect, a bone anchor assembly includes a receiver extending along a central longitudinal axis between a proximal end and an opposite distal end. The receiver includes a distal portion defining a receptacle opening at the distal end and a pair of arms extending from the distal portion along the central longitudinal axis on opposite sides of a passage that opens at opposite sides of the receiver. The receiver includes a bottom surface extending along the passage between the pair of arms and the receptacle opens into the passage through the bottom surface. The bone anchor assembly also includes a bone anchor with a distal bone engaging portion and a head at a proximal end of the distal bone engaging portion. The head includes opposite parallel side surfaces and opposite convexly curved surfaces extending between the parallel side surfaces. The receptacle of the receiver is defined by opposite parallel sides positioned along the parallel side surface of the head and the receptacle is also defined by opposite concavely curved surfaces along the convexly curved surfaces of the head so that pivoting of the receiver around the head is limited to a direction defined by a plane that is parallel to the parallel side surfaces of the head. A cap is positioned around the head and the cap includes a platform extending through the opening of the receptacle into the passage so that the cap pivots in the plane upon pivoting of the receiver about the head of the anchor. The bone anchor assembly also includes a saddle positioned in the passage of the receiver adjacent to the bottom surface of the receiver and the saddle includes a proximal support surface and a distal surface opposite the proximal support surface that contacts the platform of the cap. A connecting element extends along a central longitudinal axis and is located in the passage against the proximal support surface of the saddle and extends through the opposite sides of the receiver.

According to another aspect, a bone anchor assembly comprises a receiver extending along a central longitudinal axis between a proximal end and an opposite distal end. The receiver includes a distal portion defining a distally opening receptacle and a pair of arms extending from the distal portion along the central longitudinal axis on opposite sides of a passage that opens at opposite sides of the receiver. The receiver includes a bottom surface extending along the passage between the pair of arms and the receptacle opens through the bottom surface into the passage. The bone anchor assembly also includes a bone anchor with a distal bone engaging portion and a head at a proximal end of the distal bone engaging portion that is positioned in the receptacle with the bone engaging portion extending through the distal end of the receiver. The bone anchor assembly also includes a cap positioned in the receptacle at a proximal side of the head of the bone anchor and the cap includes a platform extending through the opening of the receptacle into the passage. The bone anchor assembly also includes a saddle positioned in the passage of the receiver adjacent to the bottom surface of the receiver that includes a proximal support surface and a distally opening receptacle opposite the proximal support surface that receives the platform. The saddle includes rails extending around the distally opening receptacle on opposite sides and opposite ends of the saddle and a distal surface in the receptacle that contacts the platform of the cap. A connecting element extending along a central longitudinal axis is located in the passage and extends through the opposite sides of the receiver. A respective one of the rails at the ends of the distal receptacle of the saddle contacts the platform of the cap when the saddle and the connecting element are pivoted in the receiver to a maximum angle from an orthogonal orientation of the central longitudinal axis of the connecting element with the central longitudinal axis of the receiver.

Related features, aspects, embodiments, objects and advantages of the present invention will be apparent from the following description.

### BRIEF DESCRIPTION OF THE DRAWINGS

FIG. 1 is a posterior elevation view of a spinal column segment with a spinal implant system engaged thereto.

FIG. 2 is an exploded perspective view of a bone anchor assembly of the spinal implant system of FIG. 1.

FIG. 3 is a longitudinal section view of the bone anchor assembly of FIG. 2 with the saddle in an orthogonal orientation relative to the connecting element and receiver.

FIG. 4 is a longitudinal section view of the bone anchor assembly in a direction orthogonal to the direction of the section of FIG. 3.

FIG. 5 is an elevation view of a bone anchor of the bone anchor assembly.

FIG. 6 is a side elevation view of the bone anchor of FIG. 5.

FIG. 7 is a top plan view of the bone anchor of FIG. 5.

FIG. 8 is an elevation view of a receiver of the bone anchor assembly of FIG. 2.

FIG. 9 is a side elevation view of the receiver of FIG. 8.

FIG. 10 is a top plan view of the receiver of FIG. 8.

FIG. 11 is a bottom plan view of the receiver of FIG. 8.

FIG. 12 is an elevation view of a cap of the bone anchor assembly of FIG. 2.

FIG. 13 is a side elevation view of the cap of FIG. 12.

FIG. 14 is a top plan view of the cap of FIG. 12.

FIG. 15 is a bottom plan view of the cap of FIG. 12.

FIG. 16 is an end elevation view of a saddle of the bone anchor assembly of FIG. 1.

FIG. 17 is a side elevation view of the saddle of FIG. 16.

FIG. 18 is a top plan view of the saddle of FIG. 16.

FIG. 19 is a bottom plan view of the saddle of FIG. 16.

FIG. 20 is a side elevation view of the bone anchor assembly with the saddle pivoted to a first maximum angular orientation in the receiver.

FIG. 21 is a frontal elevation view of the bone anchor assembly in FIG. 20 showing the receiver pivoted to a first maximum angular orientation relative to the bone anchor.

FIG. 22 is a longitudinal section view of another embodiment bone anchor assembly with the saddle pivoted in a maximum angular orientation relative to the bone anchor and receiver.

FIG. 23 is a longitudinal section view of the bone anchor assembly of FIG. 22 in a direction orthogonal to the direction of the section of FIG. 22.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices, and such further applications of the principles of the invention as illustrated herein are contemplated as would normally occur to one skilled in the art to which the invention relates.

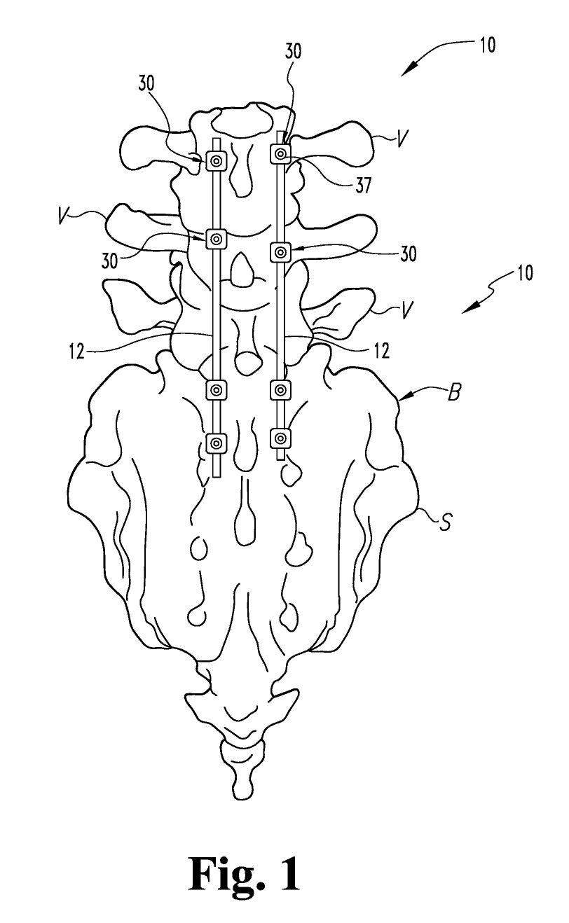

FIG. 1 illustrates a posterior spinal implant system 10 located along a spinal column of a patient. Implant system 10 generally includes several bone anchor assemblies 30 with at least one elongated connecting element 12 structured to selectively interconnect two or more bone anchors. Connecting elements 12 may be a spinal rod, plate, bar, or other elongated element having a length to extend between at least two vertebrae. Spinal implant system 10 may be used for, but is not limited to, treatment of degenerative spondylolisthesis, fracture, dislocation, scoliosis, kyphosis, spinal tumor, and/or a failed previous fusion. More specifically, in one embodiment implant system 10 is affixed to posterior elements, such as the pedicles of vertebra V, or other bones B of the spinal column segment, from a posterior approach. Bones B can include the sacrum S and/or one or more of several vertebrae V. Spinal implant system 10 can be engaged to vertebrae of one or more levels of the sacral, lumbar, thoracic and/or cervical regions of the spinal column. Other embodiments contemplate that spinal implant system 10 is engaged along other portions of the spine, such as the anterior, lateral or oblique portions of the vertebrae V. Still other embodiments contemplate applications in procedures other the spinal stabilization procedures.

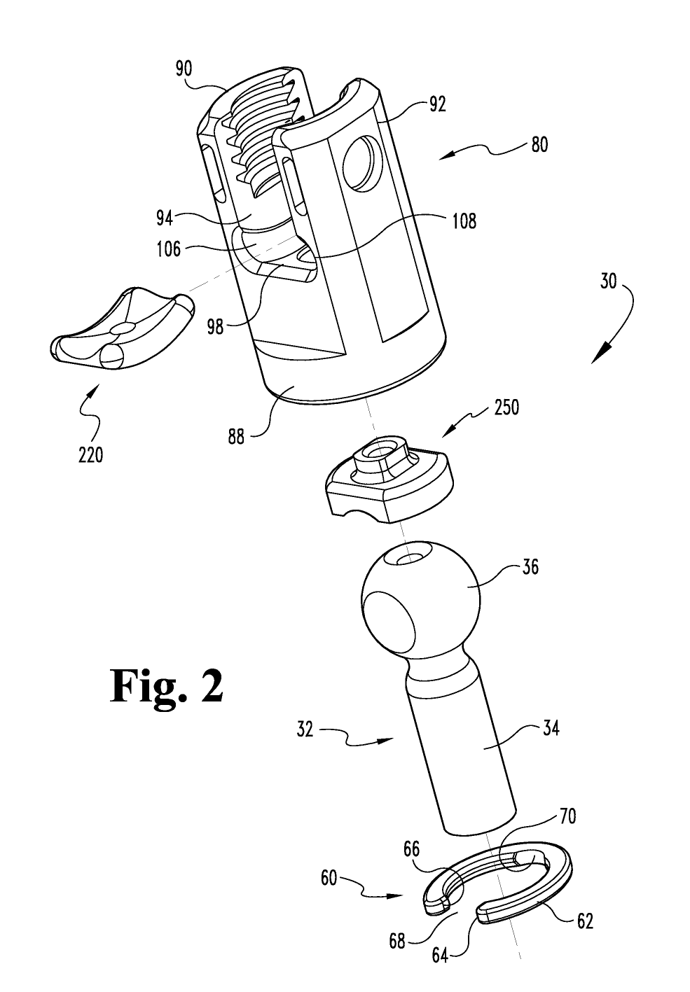

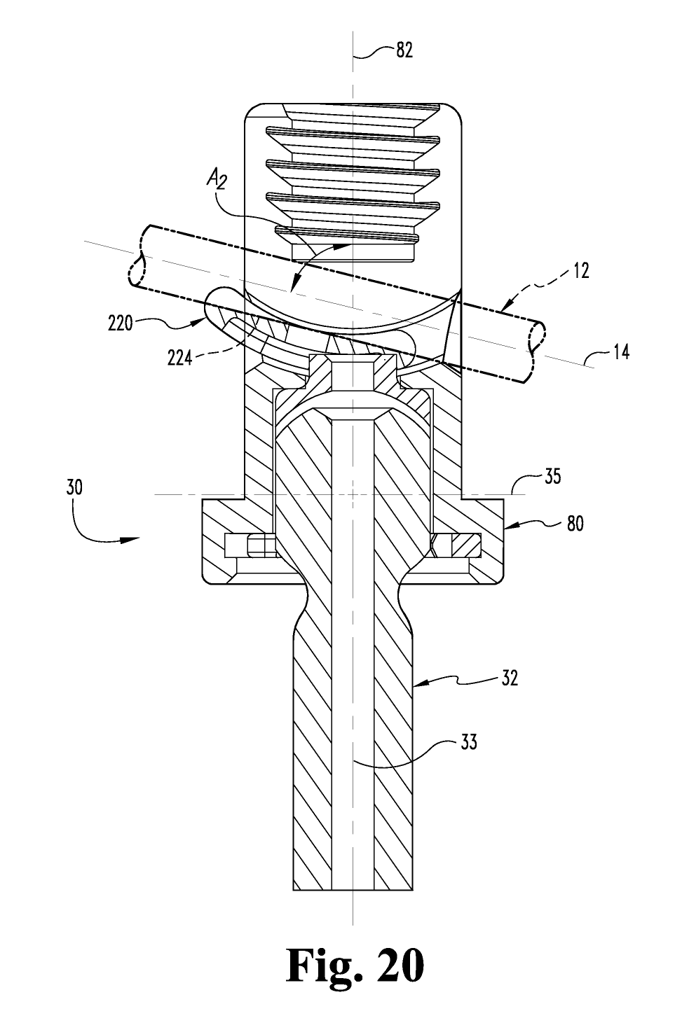

FIG. 2 shows an exploded perspective view of a bone anchor assembly 30. Bone anchor assembly 30 includes a bone anchor 32 with a distal bone engaging portion 34 and a proximal head 36. Bone anchor assembly 30 also includes a proximal receiver 80 coupled to bone anchor 32 around head 36 with a retaining member 60. Receiver 80 includes a passage 94 to receive connecting element 12 in a transverse orientation to bone anchor 32, as shown in FIGS. 1 and 20, for example. Bone anchor assembly 30 also includes a cap 250 positioned in receiver 80 on a proximal side of head 36, and a saddle 220 in passage 94 on a proximal side of cap 250. An engaging member 37 (FIG. 1) engages receive 80 and secures connecting element 12 in position in passage 94.

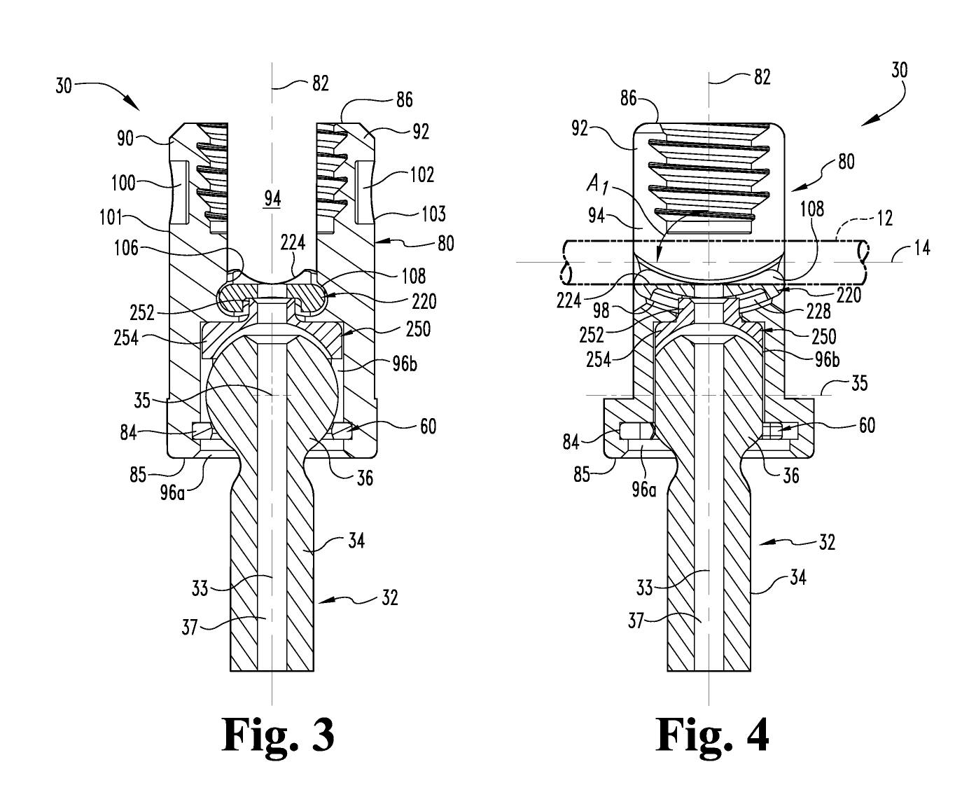

Referring further to FIGS. 3-4, there is shown longitudinal section views of bone anchor assembly 30. Bone anchor assembly 30 includes bone anchor 32 with distal bone engaging portion 34 extending distally from receiver 80 along a central longitudinal axis 33. Bone engaging portion 34 is configured for attachment to a vertebra, such as cervical, thoracic, lumbar and/or sacral vertebrae, or other bones or tissues in the body of a patient. Proximal head 36 of bone anchor 32 is positioned in a receptacle 96 of receiver 80. Retaining member 60 extends around the distal side of head 36 and is received in a groove 84 of receiver 80 that extends around receptacle 96 adjacent a distal opening of receptacle 96. Retaining member 60 prevents head 36 from passing through the distal opening of receptacle 96, while also allowing receiver 80 to pivot about head 36 in a plane parallel to opposite side surfaces of head 36, as discussed further below. Cap 250 includes a distal cup portion 254 positioned on the proximal side of head 36, and a platform 252 extending proximally from cup portion 254 into passage 94. Saddle 220 is positioned in passage 94 and around platform 252. In addition, receiver 80 includes opposite grooves 106, 108 extending along facing inner surfaces thereof. The sides of saddle 220 are positioned in grooves 106, 108 and move along passage 94 in grooves 106, 108 to various angular orientations relative to receiver 80 and bone anchor 32 in a plane defined by the central longitudinal axis 14 of connecting element 12 and central longitudinal axis 82 of receiver 80, as discussed further below.

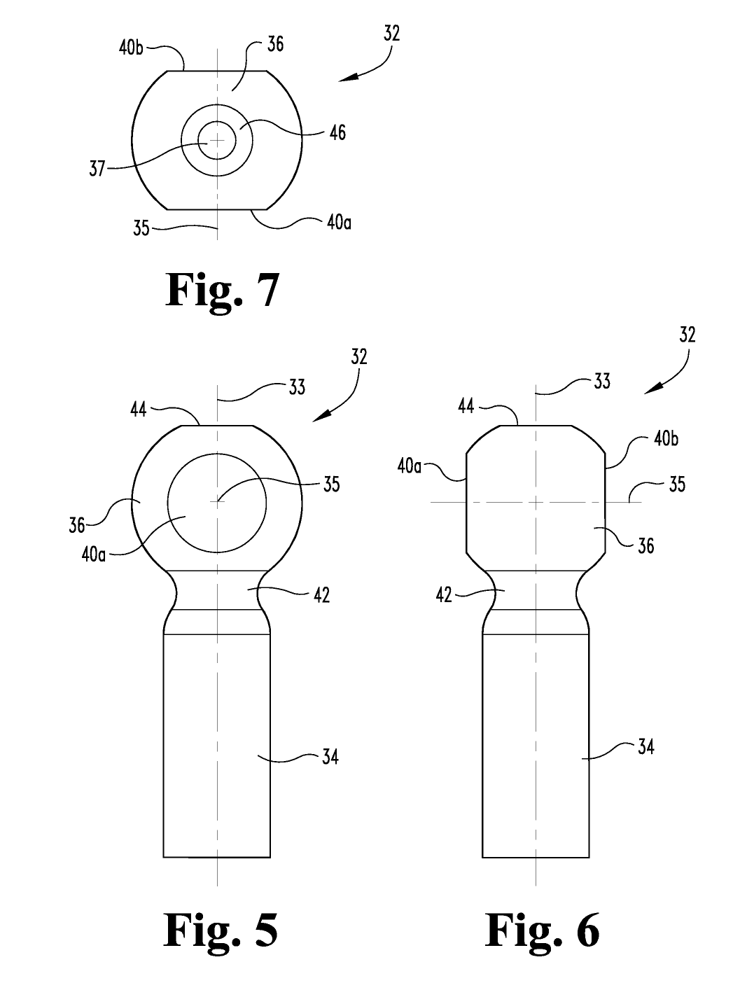

FIGS. 5-7 show additional views of bone anchor 32. Bone anchor 32 described herein can be provided with bone engaging portion 34 configured as a bone screw, vertebral hook, bone clamp, and or other suitable bone engaging arrangement. Bone anchor 32 includes a bone engaging portion 34 extending along a central longitudinal axis 33 from a proximal head 36 that is centered on central longitudinal axis 35. Bone engaging portion 34 is shown with an elongated shaft configuration, and may include one or more threads (not shown) along at least a portion thereof. The threads may be cancellous threads with the shaft sized and configured for implantation into a vertebra or other bone. The threads of bone engaging portion 34 may be self-tapping, self-drilling, continuous, intermittent, of multiple thread forms, or other appropriate configurations. Furthermore, bone anchor 32 may include a lumen 37, or be solid. Lumen 37 extends through the proximal and distal ends of anchor 32 for receipt of a guidewire and/or injection of material into the bone. One or more fenestrations may be provided along bone engaging portion 34 of bone anchor 32 that communicate with lumen 37.

Bone anchor 32 includes a transition portion 42 between bone engaging portion 34 and head 36. Transition portion 42 provides a neck between bone engaging portion 34 and head 36 around which retaining member 60 extends as shown in FIGS. 3-4. In the illustrated embodiment transition portion 42 is concavely curved to form a recess distally of head 36. In addition, retaining member 60 includes a C-shaped body 62 with opposite ends 64, 66 forming a gap 68 therebetween. A notch 70 is formed in an inner edge of body 62 opposite gap 68. Gap 68 and notch 70 facilitate moving of ends 64, 66 toward one another to reduce the outer dimension of retaining member 60 for positioning through the distal end opening of receptacle 96 and into groove 84. Retaining member 60 is resilient to spring back toward its initial configuration and engage receiver 80 in groove 84, while body 62 protrudes at least partially into receptacle 96 and below head 36 to prevent head 36 from passing through the distal opening of receiver 80. Head 36 includes an enlarged configuration with a partially spherical shape having opposite flat sides 40a, 40b that are parallel to one another. A pivot axis 35 extends through the center of sides 40a, 40b orthogonally to longitudinal axis 33. Head 36 also includes a proximal end 44 with a recess 46 extending around lumen 37.

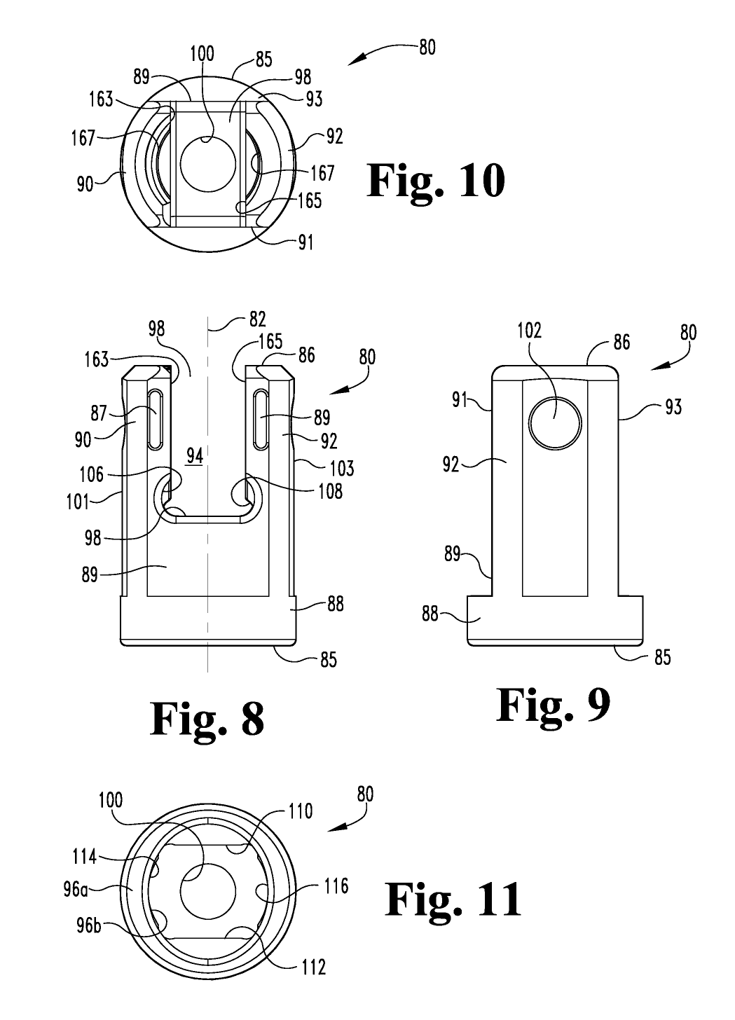

FIGS. 8-11 show further details of receiver 80. Receiver 80 includes a U-shaped body extending along central longitudinal axis 82 between a distal end 85 and a proximal end 86. Receiver 80 includes a distal connection portion 88 extending proximally from distal end 85, and a pair of arms 90, 92 extending proximally from distal connection portion 88 on opposite sides of longitudinal axis 82 to proximal end 86. Arms 90, 92 define passage 94 therebetween that opens at opposite sides 91, 93 of arms 90, 92 to receive connecting element 12 in a transverse orientation to longitudinal axis 82. Connection portion 88 includes a circular outer profile adjacent distal end 85, and distal portion 96a of receptacle 96 extends therein to form an opening at distal end 85. The groove 84 that receives retaining member 60 extends around the circular part of connection portion 88 and distal portion 96a of receptacle 96. Arms 90, 92 include outer side surfaces 101, 103, respectively, that define a circular outer surface profile that is substantially aligned with the circular outer profile of connection portion 88. The sides 91, 93 of arms 90, 92 where passage 94 opens and where the proximal part 89 extends distally from passage 94 are linear in shape to truncate the sides of the circular profile defined by arms 90, 92, defining a proximal part of receiver 80 with a footprint that defines a rectangular-like shape having linear sides and rounded ends extending proximally from and residing within the circular footprint of the distal part of receiver 80, as shown in FIG. 10.

As also shown in FIGS. 3-4, receptacle 96 also includes a proximal portion 96b that opens into passage 94 and extends from passage 94 to distal portion 96a. Arms 90, 92 also define a proximal opening 98 at proximal end 86 that extends along arms 90, 92 to passage 94. Engaging member 37 is engaged to receiver 80 through the proximal end opening 98 to contact connecting element 12 in passage 94 and secure connecting element 12 against saddle 220. Arms 90, 92 each include a circular recess 100, 102 in the outer side surface 101, 103, respectively, that face opposite directions from one another. Arm 90 also includes oblong recesses 87 in each of the opposite side surfaces 91, 93 thereof. Arm 92 similarly includes oblong recesses 89 in each of the opposite side surfaces 91, 93 thereof. The recesses 87, 89 provide locations in which various tools and instrumentation can be engaged and mounted to receiver 80 to facilitate implantation and maneuvering of bone anchor 30 and connecting element 12 in the patient. Side surfaces 91, 93 each are elongated in a longitudinal direction in a parallel orientation to longitudinal axis 82, and extend between the respective outer side surface 101, 103 to the respective inner surface 163, 165 of arms 90, 92 in an orthogonal orientation to longitudinal axis 82. Each of the inner surfaces 163, 165 includes a central concavely curved portion and linear opposite end portions between the end surfaces 91, 93 of the respective arm 90, 92. The central concave portion of inner surface 163, 165 defines a thread profile 167 to threadingly engage engaging member 37. Each thread profile 167 extends along longitudinal axis 82 from proximal end 86 of arms 90, 92 to a location adjacent to passage 94 in receiver 80.

Receiver 80 includes a concavely curved bottom surface 98 along the distal side of passage 94 that is linear between inner surfaces 163, 165 of arms 90, 92 and concavely curved between opposite sides 91, 93. Receptacle 96 opens through bottom surface 98 into passage 94. In addition, each of the arms 90, 92 includes groove 106, 108, respectively, formed in the respective inner surface 163, 165 thereof adjacent to bottom surface 98. Each groove 106, 108 extends from and opens through the side surfaces 91, 93 of the respective arm 90, 92. Each groove 106, 108 is curved between the respective side surfaces of the arm in which it is formed so that the middle of the curved groove at longitudinal axis 82 is located more distally than the opposite ends of the groove at sides 91, 93. Grooves 106, 108 extend from the middle portion thereof so that the opposite ends of grooves 106, 108 are spaced proximally from bottom surface 98 where the groove exits at the opposite end surfaces 91, 93 of the respective arm 90, 92. Saddle 220 is sized and configured to slide in grooves 106, 108 to allow for uni-planar adjustment of connecting element 12 and saddle 220 in and relative to receiver 80.

As shown in FIGS. 10 and 11, receptacle 96 includes a circular opening through bottom surface 98 that is defined by a lip 100 extending around the proximal side of receptacle 96. As previously set forth, spacer 250 includes platform 252 sized to extend through the circular opening to contact lower portion of saddle 220 positioned along bottom surface 98. The proximal portion 96b of receptacle 96 includes a rectangular shape with linear elongate sides 110, 112 and shorter rounded ends 114, 116 extending between elongate sides 110, 112. Head 36 of anchor 32 is positioned in receptacle 96 with planar sides 40a, 40b oriented toward a respective one of the linear sides 110, 112 and the spherically shaped portion of head 36 oriented toward rounded ends 114, 116. As discussed further below, receiver 80 pivots around pivot axis 35 in a plane that is parallel to planar sides 40a, 40b of head 36.

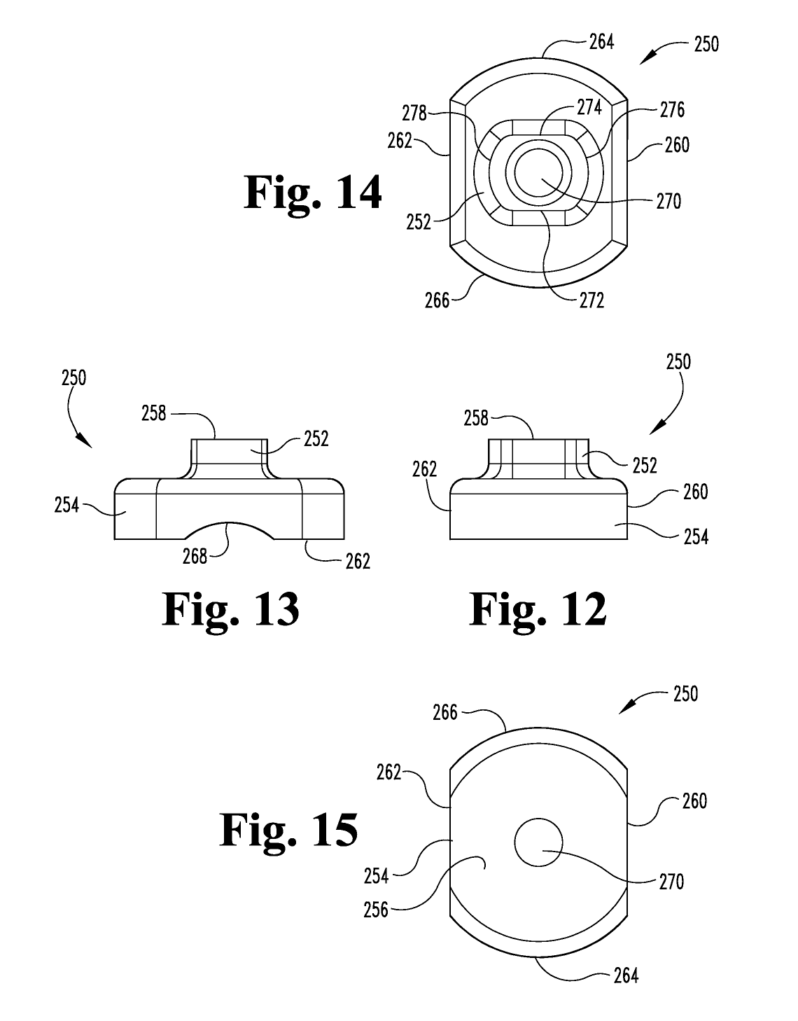

Referring collectively to FIGS. 12-15, cap 250 is shown. Cap 250 includes a proximal platform 252 projecting proximally from a distal cup portion 254. Cup portion 254 defines a partially spherical cavity 256 with linear sides 260, 262 and rounded ends 264, 266 that correspond to the outer profile of head 36 of anchor 32. Platform 252 includes an oblong shape that tapers proximally to a proximal end surface 258. The oblong shape is oriented with elongate sides 272, 274 toward the linear sides 260, 262 of cup portion 254 and thus oriented toward the parallel side surfaces 40a of head 36 when positioned on anchor 32. Cap 250 pivots on head 36 in a plane that is parallel to side surfaces 40a as receiver 80 pivots in the plane parallel to side surfaces 40a. Linear sides 260, 262 also each include a concave recess 268 in a distal surface thereof that extends along and follows the proximal surface curvature of head 36 along sides 40a so that cup portion 254 does not extend along sides 40a, allowing sides 40a to be positioned against linear sides 110, 112 of receptacle 96. Thus, the interface between receiver 80 and head 36 controls the uni-planar pivoting motion of receiver 80 without interference from cap 250. A circular hole 270 extends from cavity 256 through proximal surface 258 of platform 252.

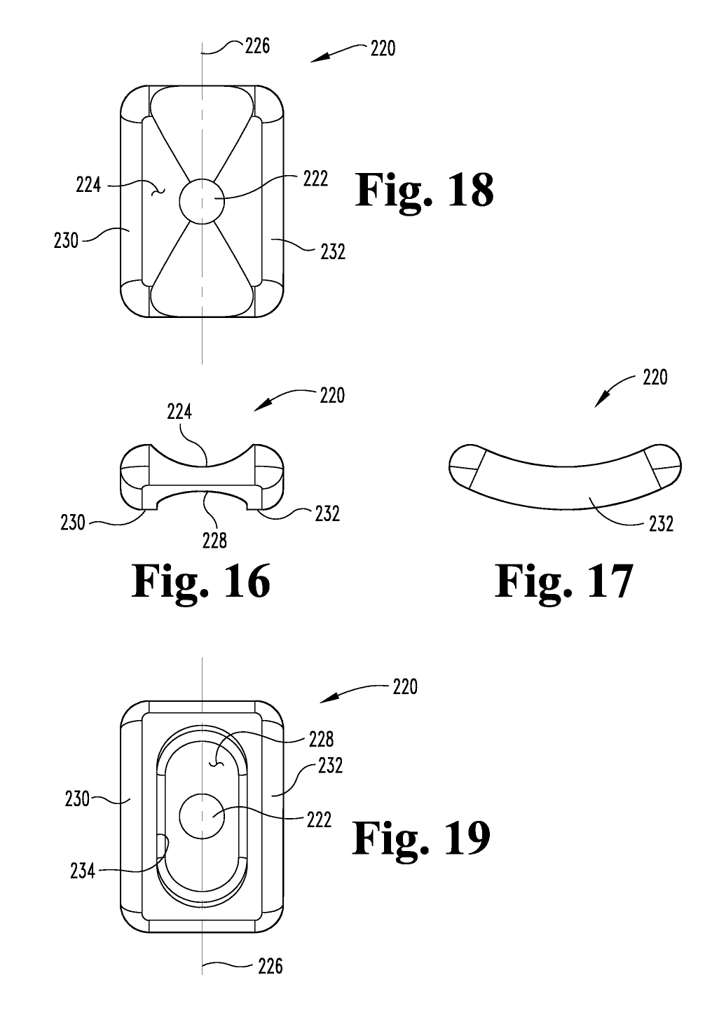

FIGS. 16-19 show various view of saddle 220. Saddle 220, as also shown in FIGS. 3-4, is positioned in passage 94 of receiver 80 between connecting element 12 and cap 250. Saddle 220 includes a body with a rectangular shape when saddle 220 is viewed in a proximal to distal direction, as shown in FIGS. 18-19. Saddle 220 extends along a longitudinal axis 226 between opposite ends, and includes a circular hole 222 extending through a center thereof between upper and lower surfaces thereof. Hole 222 is alignable with hole 270 of cap 250 and lumen 37 of bone anchor 32. Saddle 220 includes a proximal support surface 224 against which connecting element 12 is positioned. Proximal support surface 224 is linear in a direction paralleling longitudinal axis 226 as shown in FIG. 4 and is concavely curved orthogonally to longitudinal axis 226 as shown in FIGS. 3 and 16. The shape of proximal support surface 224 matches the shape of the portion of the outer surface of connecting element 12 positioned thereagainst.

Saddle 220 includes a distal surface 228 opposite proximal support surface 224. Distal surface 228 is convexly curved along longitudinal axis 226 as shown in FIG. 4. Distal surface 228 contacts and is supported by platform 252 of cap 250. The convexly curved distal surface 228 facilitates pivoting movement of saddle 220 in the plane that includes longitudinal axis 82 of receiver 80 and longitudinal axis 14 of connecting element 12. In addition, saddle 220 includes side rails 230, 232 extending along opposite sides of saddle 220 that are received in and translate along respective ones of the grooves 106, 108 of receiver 80. Rails 230, 232 maintain saddle 220 within receiver 80 along a path defined by grooves 106, 108 and prevent saddle 220 from pivoting or twisting to an undesired orientation in receiver 80. Rails 230, 232 project distally also extend along the ends of saddle 220 to enclosed distal surface 228 and define a distal receptacle 234 that is elongated along axis 226 and receives platform 252 therein. The oblong platform 252 is oriented so its elongate sides 272, 274 extend in the direction along axis 226 in which receptacle 234 is also elongated. When saddle 220 is pivoted to a maximum angular orientation in receiver 80, one of the ends 276, 278 of platform 252 contacts an adjacent end of receptacle 234 to limit or prevent further pivoting of saddle 220 beyond the maximum angular orientation in receiver 80.

Referring now to FIG. 20, saddle 220 supports connecting element 12 in receiver 80 and pivots in a plane defined by the central longitudinal axis 14 of connecting element 12 and central longitudinal axis 82 of receiver 80. Saddle 220 supports connecting element 12 and maintains a proximal support surface of saddle 22 in contact with connecting element 12 at various orientations of longitudinal axis 14 relative to longitudinal axis 82 that vary from an orthogonal orientation A1, such as shown in FIG. 4, to maximum angular orientation A2, as shown in FIG. 20, where an end of receptacle 234 contacts platform 252. It is understood that saddle 220 is also pivotable in the opposite direction in receiver 80 to accommodate connecting element 12 angled distally to proximally in the left side to right side direction. In one embodiment, angle A2 is 60 degrees from the orthogonal orientation of connecting element 12. Other embodiments contemplate angle A2 ranging from 90 degrees to about 45 degrees. The orientation of connecting element 12 and saddle 220 can vary at any angle A2 relative to longitudinal axis 82 of receiver 80 while the orientation between longitudinal axis 82 of receiver 80 and longitudinal axis 33 of bone anchor 32 is maintained in an aligned, linear orientation when viewed in a direction looking orthogonally at the plane defined by longitudinal axis 33 of anchor 32 and longitudinal axis 12 of connecting element 12.

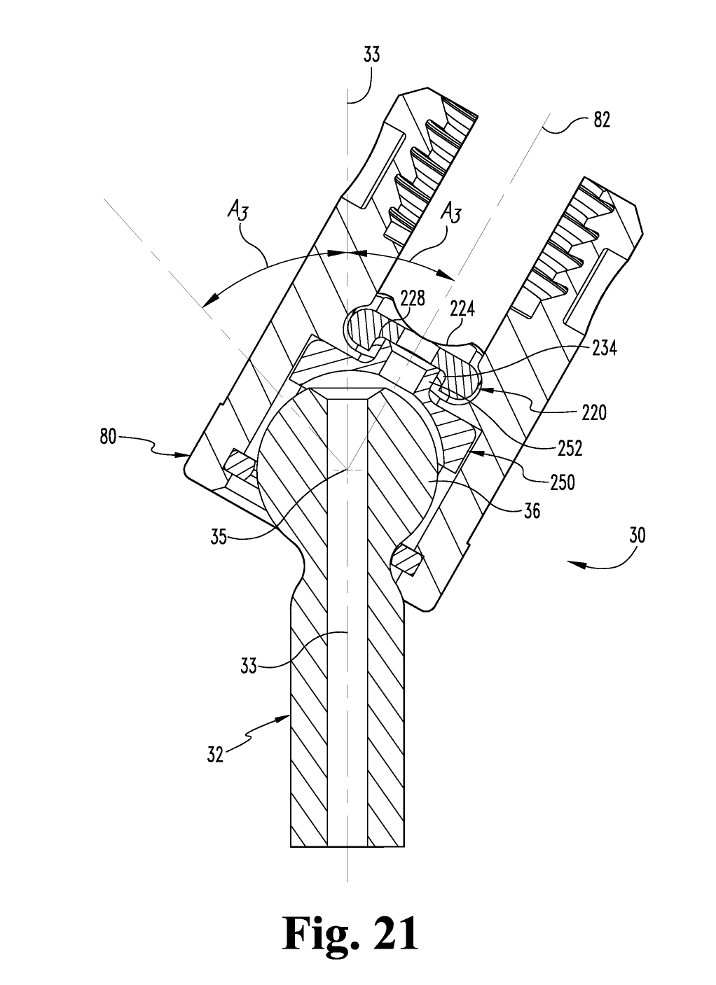

In addition, as shown in FIG. 21, receiver 80 pivots about head 36 around pivot axis 35 to vary the angular orientation of longitudinal axis 82 of receiver 80 relative to longitudinal axis 33 of bone anchor 32 in a single plane that is orthogonal to the plane in which saddle 220 pivots or translates in receiver 80. In a first orientation, longitudinal axis 82 is aligned with and co-linear with longitudinal axis 33 so that connecting element 12 is centered relative to longitudinal axis 33. Receiver 80 is pivotable about pivot axis 35 to vary the angular orientation of longitudinal axis 82 from longitudinal axis 33 up to a maximum angle A3, allowing receiver 80 to receive connecting element 12 in an offset relation to longitudinal axis 33. In one embodiment, angle A3 can range from 0 degrees up to about 30 degrees in either direction from a linear extension of longitudinal axis 33. Other embodiments contemplate angle A3 ranging from 0 degrees to about 45 degrees.

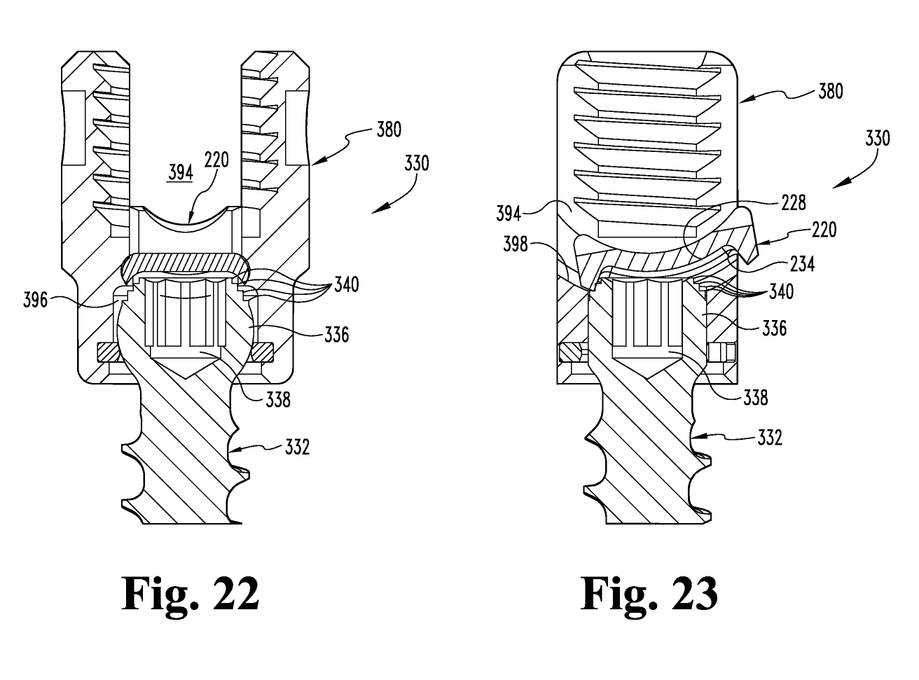

FIGS. 22 and 23 show another embodiment bone anchor assembly 330 with a receiver 380 mounted to a bone anchor 332. Saddle 220 is positioned in receiver 380 so that a connecting element 12 can be positioned against a proximal support surface of saddle 220, in the same manner discussed above with respect to bone anchor assembly 30. Receiver 380 differs from receiver 80 in that receptacle 396 of receiver 380 and the opening of receptacle 396 through bottom surface 398 is sized to allow the proximal side of head 336 of bone anchor 332 to extend therethrough and contact the distal surface of saddle 220. Therefore, a cap between head 336 and saddle 220 is eliminated. Head 336 includes an outer profile or footprint like head 36 of bone anchor 32 so that receiver 380 pivots around head 336 in a plane that is orthogonal to the longitudinal axis 14 of connecting element 12 in passage 394. Head 336 includes a plurality of ridges 340 extending around its proximal side that extend into passage 394 of receiver 380 and contact distal surface 228 of saddle 220 in receptacle 234 of saddle 220. When saddle 220 is compressed against ridges 340, ridges 340 bite into saddle 220 and limit or prevent further movement of saddle 220 in receiver 380. As shown in FIG. 23, when saddle 220 is pivoted to a maximum angular orientation in receiver 380, one of the ends of receptacle 234 contacts the ridges 340 at the adjacent side of head 336 to prevent further movement of saddle 220 and fix saddle 220 in position in receiver 380.

The bone anchor assemblies discussed herein allow adjustment of the angle of the saddle and thus the angle of the connecting element extending through the saddle in a single plane defined by the longitudinal axis of the connecting element and the longitudinal axis of the receiver. The anchor assemblies also allow adjustment of the receiver relative to the anchor member in a plane or direction that is transversely oriented to the plane in which the saddle adjusts in the receiver. The bone anchor assemblies provide a two-piece construct for the receiver and the bone anchor that forms a rigid or semi-rigid bone anchor assembly when the receiver is assembled with the bone anchor. The two piece construct allows the receiver and bone anchor to be comprised of different materials suitable for the expected loading of the components. For example, the receiver can be made from a higher strength material than the material for the bone anchor so that the splaying and other deformations of the receiver can be limited by the higher strength material and so that the side of the receiver can be minimized to limit intrusiveness into the surrounding tissue post-implantation.

Materials for the anchors, receivers, saddles and engaging members disclosed herein can be chosen from any suitable biocompatible material, such as titanium, titanium alloys, cobalt-chromium, cobalt-chromium alloys, or other suitable metal or non-metal material. Connecting element 12 can be made from the same material as one or more of the components of the anchor assembly to which it is engaged, or from a different material. For example, connecting element 12 can be made from PEEK, plastic, titanium or titanium alloy, cobalt-chrome, composite material, or other material that is the same or different from the material of one or more components of the anchor assembly to which is engaged. The anchor assemblies can be sized for placement at any level of the spine and for engagement with any bony portion of the spine. In one particular embodiment, the anchor assemblies are engaged to pedicles of the vertebrae. Of course, it is understood that the relative size of the components of the anchor assemblies can be modified for the particular vertebra(e) to be instrumented and for the particular location or structure of the vertebrae to which the anchor assembly will be engaged.

Although various embodiments have been described as having particular features and/or combinations of components, other embodiments are possible having a combination of any features and/or components from any of embodiments as discussed above. As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the terms “proximal” and “distal” refer to the direction closer to and away from, respectively, an operator (e.g., surgeon, physician, nurse, technician, etc.) who would insert the medical implant and/or instruments into the patient. For example, the portion of a medical instrument first inserted inside the patient's body would be the distal portion, while the opposite portion of the medical device (e.g., the portion of the medical device closest to the operator) would be the proximal portion.

While the invention has been illustrated and described in detail in the drawings and foregoing description, the same is to be considered as illustrative and not restrictive in character, it being understood that all changes and modifications that come within the spirit of the invention are desired to be protected.

What is claimed is:

1. A bone anchor assembly, comprising:
a receiver extending along a central longitudinal axis between a proximal end and an opposite distal end, said receiver including a distal portion defining a receptacle opening at said distal end and a pair of arms extending from said distal portion along said central longitudinal axis on opposite sides of a passage, said receiver including a bottom surface extending along said passage between said pair of arms, wherein said passage opens at opposite sides of said receiver between said pair of arms and said receptacle opens into said passage through said bottom surface;
a bone anchor including a distal bone engaging portion extending along a longitudinal axis and a head at a proximal end of said distal bone engaging portion, said head being positioned in said receptacle;
a saddle positioned in said passage of said receiver adjacent to said bottom surface of said receiver, said saddle including a proximal support surface and a distal surface opposite said proximal support surface; and
a connecting element extending along a central longitudinal axis thereof, said connecting element being located in said passage and extending through said opposite sides of said receiver, wherein said saddle engages said receiver and is limited to movement in said receiver in a first plane defined by said central longitudinal axis of said receiver and said central longitudinal axis of said connecting element while said receiver is pivotal about said head of said bone anchor in a second plane that is orthogonal to said first plane in which said saddle pivots to vary an angular orientation of said central longitudinal axis of said receiver relative to said longitudinal axis of said bone engaging portion.

2. The bone anchor assembly of claim 1, wherein:
said saddle is movable in said receiver so that said support surface parallels said longitudinal axis of said connecting element in orientations of said longitudinal axis of said connecting element that vary up to 30 degrees from an orthogonal orientation of said longitudinal axis of said connecting element with said central longitudinal axis of said receiver; and
said receiver is pivotable about said head of said bone anchor so that said central longitudinal axis of said receiver varies up to 30 degrees from a linear orientation with said longitudinal axis of said bone anchor.

3. The bone anchor assembly of claim 1, wherein:
said pair of arms include inner surfaces facing one another on opposite sides of said passage;
said inner surfaces each include a groove formed therein between opposite sides of a respective one of said pair of arms so that each of said grooves includes a most distal portion at said central longitudinal axis and is curved proximally from said most distal portion toward said opposite sides of said respective arm; and
said saddle including at least one rail on each side thereof that is positioned in a respective one of said grooves, said rails being slidably movable along said respective one of said grooves through said opposite sides of said arms.

4. The bone anchor assembly of claim 1, further comprising a cap positioned between said head of said bone anchor and said saddle, said cap including a distal cup portion around a proximal side of said head and a platform extending proximally from said cup portion into contact with said distal surface of said saddle.

5. The bone anchor assembly of claim 4, wherein said head of said anchor includes opposite parallel side surfaces and convexly curved ends extending between said parallel side surfaces, and said receptacle includes opposite, linear sides positioned along respective ones of said parallel side surfaces of said head and concavely curved ends positioned along said convexly curved ends of said head.

6. The bone anchor assembly of claim 5, wherein said receiver includes a lip between said receptacle and said bottom surface of said receiver, said lip defining a circular opening between said passage and said receptacle, and said platform extends through said circular opening to contact said distal surface of said saddle.

7. The bone anchor assembly of claim 6, wherein:
said saddle includes distally opening receptacle and said distal surface is located in said receptacle; and
said platform of said cap is received in said receptacle of said saddle; and
said receptacle of said saddle includes opposite ends that contact said platform to limit movement of said saddle in said first plane when said saddle and said connecting element are oriented in said receiver at a maximum angle from an orthogonal orientation of said central longitudinal axis of said connecting element with said central longitudinal axis of said receiver.

8. The bone anchor assembly of claim 5, wherein said distal cup portion of said cap includes opposite parallel sides and opposite curved ends extending between said parallel sides, and said curved ends are positioned along said convexly curved ends of said head of said anchor member and said parallel sides of said cup portion each include a concave recess in a distal surface thereof so that a distal end of said parallel sides of said cup portion are positioned proximally of said parallel side surfaces of said head of said anchor in said receptacle.

9. The bone anchor assembly of claim 1, wherein said receptacle of said receiver includes a distal opening through which said bone engaging portion of said bone anchor extends and a retaining member extending around said distal opening that contacts a distal side of said head to prevent said head from passing through said distal opening of said receptacle of said receiver.

10. The bone anchor assembly of claim 1, wherein said receiver includes a distal part that defines a circular outer surface profile and a proximal part extending proximally from said distal part to said proximal end of said receiver, said proximal part including opposite circular portions extending around outer surfaces of said pair of arms and opposite linear portions extending between said circular portions, wherein said linear portions extend distally from said bottom surface along said opposite sides of said receiver where said connecting element extends from said passage to said distal part of said receiver.

11. The bone anchor assembly of claim 1, wherein a proximal end of said head of said bone anchor includes a plurality of ridges extending around said head and said head is positioned in said receiver so that said ridges contact said distal surface of said saddle, wherein said ridges bite into said saddle to fix said saddle in position in said receiver against said proximal support surface of said saddle.

12. A bone anchor assembly, comprising:
a receiver extending along a central longitudinal axis between a proximal end and an opposite distal end, said receiver including a distal portion defining a receptacle opening at said distal end and a pair of arms extending from said distal portion along said central longitudinal axis on opposite sides of a passage with said passage opening at opposite sides of said receiver, said receiver including a bottom surface extending along said passage between said pair of arms, wherein said receptacle opens into said passage through said bottom surface;
a bone anchor including a distal bone engaging portion and a head at a proximal end of said distal bone engaging portion, said head including opposite parallel side surfaces and opposite convexly curved surfaces extending between said parallel side surfaces, said receptacle being defined by opposite parallel sides positioned along said parallel side surface of said head and said receptacle further being defined by opposite concavely curved surfaces along said convexly curved surfaces of said head, wherein pivoting of said receiver around said head is limited to a direction defined by a plane that is parallel to said parallel side surfaces of said head;
a cap positioned around said head, said cap including a platform extending through said opening of said receptacle into said passage, wherein said cap pivots in said plane upon pivoting of said receiver about said head of said anchor;
a saddle positioned in said passage of said receiver adjacent to said bottom surface of said receiver, said saddle including a proximal support surface and a distal surface opposite said proximal support surface, said distal surface contacting said platform of said cap; and
a connecting element extending along a central longitudinal axis, said connecting element being located in said passage against said proximal support surface of said saddle and extending through said opposite sides of said receiver.

13. The bone anchor assembly of claim 12, wherein said saddle engages said receiver and is limited to movement relative to said receiver in a single plane defined by said central longitudinal axis of said receiver and said central longitudinal axis of said connecting element.

14. The bone anchor assembly of claim 12, wherein said opening of said receptacle into said passage is circular and said platform is oblong with a long axis of said platform extending along said central longitudinal axis of said connecting element.

15. The bone anchor assembly of claim 14, wherein said saddle includes a distally opening receptacle and said distal surface is located in said receptacle, said platform being received within said distally opening receptacle of said saddle.

16. The bone anchor assembly of claim 15, wherein:
said receiver include an inner surface along each arm facing said passage and each inner surface includes a groove formed therein that extends along said passage between said opposite sides of said receiver;
said saddle includes opposite rails extending along said saddle around said distally opening receptacle, said opposite rails being position in respective ones of said grooves, said saddle translating along said grooves and through said opposite sides of said receiver as said saddle pivots in said receiver; and
said distal surface of said saddle slides on said platform of said cap as said saddle pivots in said receiver.

17. A bone anchor assembly, comprising:
a receiver extending along a central longitudinal axis between a proximal end and an opposite distal end, said receiver including a distal portion defining a receptacle opening at said distal end and a pair of arms extending from said distal portion along said central longitudinal axis on opposite sides of a passage with said passage opening at opposite sides of said receiver, said receiver including a bottom surface extending along said passage between said pair of arms, wherein said receptacle opens through said bottom surface into said passage;
a bone anchor including a distal bone engaging portion and a head at a proximal end of said distal bone engaging portion, said head being positioned in said receptacle with said bone engaging portion extending through said distal end of said receiver;
a cap positioned in said receptacle at a proximal side of said head of said bone anchor, said cap including a platform extending through said opening of said receptacle into said passage;
a saddle positioned in said passage of said receiver adjacent to said bottom surface of said receiver, said saddle including a proximal support surface and a distally opening receptacle opposite said proximal support surface receiving said platform, said saddle including rails extending around said distally opening receptacle on opposite sides and opposite ends of said saddle, said saddle including a distal surface in said receptacle contacting said platform of said cap; and
a connecting element extending along a central longitudinal axis, said connecting element being located in said passage and extending through said opposite sides of said receiver, wherein a respective one of said rails at said ends of said distal receptacle of said saddle contacts said platform of said cap when said saddle and said connecting element are pivoted in said receiver to a maximum angle from an orthogonal orientation of said central longitudinal axis of said connecting element with said central longitudinal axis of said receiver.

18. The bone anchor assembly of claim 17, wherein said saddle engages said receiver and is limited to movement relative to said receiver in a single plane defined by said central longitudinal axis of said receiver and said central longitudinal axis of said connecting element.

19. The bone anchor assembly of claim 18, wherein:
said head of said anchor includes opposite parallel side surfaces and convexly curved end surfaces extending between said parallel side surfaces, and said receptacle of said receiver includes opposite, linear sides positioned along respective ones of said parallel side surfaces of said head and concavely curved ends positioned along said convexly curved end surfaces of said head; and
said saddle engages said receiver and is limited to movement in said receiver in said single plane while said receiver is pivotal about said head of said bone anchor in a direction that parallels said parallel side surfaces of said head and said direction is orthogonal to said single plane in which said saddle pivots to vary said first orientation of said central longitudinal axis of said receiver relative to said central longitudinal axis of said bone engaging portion.

20. The bone anchor assembly of claim 19, wherein said cap include a distal cup portion having opposite parallel sides and opposite curved ends extending between said parallel sides, and said curved ends are positioned along said convexly curved ends of said head of said anchor member and said parallel sides of said cup portion each include a concave recess in a distal surface thereof so that a distal end of said parallel sides of said cup portion are positioned proximally of said parallel side surfaces of said head of said anchor in said receptacle.
